(19) United States
(12) Patent Application Publication
(10) Pub. No.: US 2006/0247578 A1
(43) Pub. Date: Nov. 2, 2006

(54) Powered automatic injection device

(76) Inventors: Andrew Arguedas, Minneapolis, MN (US); Wilbur Eidson, Minneapolis, MN (US)

Correspondence Address: Janet P. Schafer;SCHAFER LAW OFFICE, P.A., 727 Torchwood Circle, New Brighton, MN, 55112-2538, US

(21) Appl. No.: 10/916,069

(22) Filed: Aug. 11, 2004

Related U.S. Application Data
(60) Provisional application No. 60/494,213, filed on Aug. 11, 2003.

(51) Int. Cl. A61M 5/00

(52) U.S. Cl. 604/181; 604/61

(57) ABSTRACT

An automatically controlled medicament delivery system having a hand-held gun-shaped device with a handgrip, the device of unitary construction. The device has an internal motor that is powered by connection to a power source, such as AC current or DC battery, a pair of limit switches which controls the administration of the medicament or implant by limiting movement of a helical gear that moves a fitting in response to being powered by the internal motor, depressing the trigger on the gun-shaped device initiates the internal motor that powers the helical gear that in turn delivers the medicament to the individual animal. Additionally, the device has indicator lights that signal the operator when the injection device is ready to deliver the medicament, when medicament is being delivered, when medicament has been delivered, and when the helical gear is being returned to the original position readying the injection device for the next delivery of medicament. Safety switches, controlled by the user, stop delivery of the medicament when that medicament is being delivered in error. The device has optional agencies of delivering the medicament, these agencies being by a needle or spray nozzle in response to electric motor driving a helical gear transferring rotational motion from the electric motor into linear motion through a coupling attached to the helical gear.

### BACKGROUND

This invention relates to veterinary delivery devices for delivering medicaments, including pharmaceuticals or vaccines, to a plurality of poultry or other animals. In particular, this invention relates to a portable electrically powered veterinary delivery system for reliably providing a precise amount of pharmaceuticals, vaccines, or implantable device rapidly to a plurality of fowl, porcine, ovine, bovine, piscine, or other animals.

Pox experienced by chickens and other fowl is a painful, fatal disease which results in damaging the poultry crop for the farmer.

Numerous injection devices have been provided in prior art that are adapted to include a manually operated plunger, needle, or push-rod. A hand-held syringe, having a barrel and manually operated plunger, has been used to administer vaccines, antibiotics and other biological products. A problem with this method is that the accuracy of the dose is dependent on the manual strength and attention of the operator. When injecting large numbers of birds or other animals, the operator's hands become fatigued resulting in inaccurate doses being delivered to the bird or other animal being injected. An additional prior method of vaccinating birds holding a bottle of vaccine in one hand and a “pox needle”, a solid needle with an orifice formed therein, in the other, then the operator would have to spread the wing of the bird, dip the needle in the bottle, stab the bird all while trying to avoid self injection.

An additional method of vaccination birds for pox was with a device that required an operator to pour the vaccine into a bottle with a “pox needle” inside, The device was similar to a syringe but without a plunger. The device had a spring-loaded handle that was connect to the “pox needle”. When the handle was compressed, the needle would be pushed out through a gasket in the tip of the device. For an operator to deliver a dose to a bird , the operator would have to compress the spring-loaded handle until the orifice on the needle passed through the bird's wing web. This method caused fatigue to the operator's hands when vaccination large numbers of birds.

An additional problem has been accidental injury to the operator as a result of either movement of the bird or other animal during the injection process. Injection into a human of certain veterinary products can cause permanent injury that may result in amputation. An additional problem is keeping track of the inoculated animals so as not to administer a double dose, or skip an animal inadvertently. Because of the short shelf-life of the vaccine, being able to quickly administer vaccine to large numbers of birds or other animals is important. The vaccine has such a short shelf-life that once opened the poxer vaccine needs to be used within one hour.

While these units may be suitable for the particular purpose, which they address, they would not be as suitable for the purpose of the present invention as hereinafter described.

### SUMMARY

The present invention is directed to and economic, automatically controlled medicament delivery system having a hand-held gun-shaped device with a handgrip, the device of unitary construction that delivers a precise amount of medicament to an individual animal within a plurality of animals in a rapid manner. The device has an internal motor that is powered by connection to a power source, such as AC current or DC battery, a pair of limit switches which controls the administration of the medicament or implant by limiting movement of a helical gear that moves a fitting in response to being powered by the internal motor. The fitting having the “poxer needle” mounted thereon. Depressing the trigger on the gun-shaped device initiates the internal motor that powers the helical gear that in turn drives the fitting with a needle mounted thereon and delivers the medicament to the individual animal. Additionally, the device has indicator lights that signal the operator when the injection device is ready to deliver the medicament, when medicament is being delivered, when medicament has been delivered, and when the helical gear is being returned to the original position readying the injection device for the next delivery of medicament. Safety switches, controlled by the user, stop delivery of the medicament when that medicament is being delivered in error. The device has optional agencies of delivering the medicament, these agencies being by a needle or spray nozzle in response to electric motor driving a helical gear transferring rotational motion from the electric motor into linear motion through a coupling attached to the helical gear.

An automatic veterinary medicament delivery system that satisfies these needs for delivery of precise amounts of medicament or implantable devices to a plurality of individual animals or fowl in a rapid manner. The medicament includes any fluid products for immunizing and/or treatment. The device can also be used for subcutaneous implantation of medicaments or identification devices. This device provides a rapid and consistent one-handed administration of the medicament or implantable device, without fatigue-affected changes in the amount of medicament delivered, to large numbers of fowl, porcine, ovine, bovine, piscine, or other animals.

An automatic veterinary delivery system having features of the present invention comprises a hand-held injection device gun having a precise electrical control for quickly delivering numerous accurate doses of medicament or identification system, delivered by injection or implantation. The veterinary delivery system includes hand-held injection injection device, or implantation devise, having a motor and a pair of electrical limit switches that, by means of switching polarities of the motor, control a fitting that effectuates the administration of the injectable, other medicament or implantable device. The veterinary delivery system also includes a push-button trigger, signal lights, and safety switch all mounted for use on the hand-held injection injection device.

A hand-held unit provides a “poxer needle”, a single solid needle that includes an orifice formed in the side thereof for the administration for a small preset dose to be administered though the wing web of poultry. To deliver the dose, the needle has to travel through the wing web of the bird, leaving the residue of the vaccine on/in the bird's wing. The size of the orifice controls the amount of the dosage. A hand-held unit consists of a delivery system that is self-priming to automatically administer a preset dose though a hollow needle. A hand-held unit consists of a delivery system for the administration of implantation of medicament, including but not exclusive to the ear of cattle or other animals. A hand-held unit consists of a delivery system that administers a preset dose of medicament by means of spraying the medicament through a spray nozzle, into the respiratory system of animals These hand-held unit has an emergency stop feature that pauses or terminates the delivery of the medicament or implantable device in process in case of an undesired administration. To increase operator safety and control, if needed the operator can instantaneously stop the current injection and if needed terminate the injection all by releasing the safety switch located on the handle. The emergency stop feature will instantaneously reverse motion of the push rod retracting completely getting ready for the next injection. This emergency stop action is easily done by simply releasing the trigger safety switch located on the handle.

The pressure-sensitive safety switch adjacent to the needle initiates forward motion of the motor. This feature also deters accidental self-injection. Injection cannot take place until the needle is fully inserted, thus enabling the operator to withdraw an accidental stab prior to injection taking place. The goal of this particular system is operator safety. Self-injection is a very serious accident among vaccinating crews.

The delivery system is powered by a compact, rechargeable DC battery or an AC/DC power converter for sustained operation reducing fatigue and the likelihood of repetitive stress injury on the operator.

The hand-held units have four LED's indicating to the mode to the operator as follows: an amber LED indicating mode one power on motion stopped ready to inject; a green LED indicating mode two power on motion forward injection in progress; a blue LED indicating mode three power on motion stopped injection complete; a red LED indicating mode four power on motion reversed injection priming.

The hand-held unit includes a light below the cylinder indicating to the operator the presence of serum inside the cylinder. Hand-held unit is optionally equipped with a dye marking system that automatically marks an injection site when the injection is completed.

The hand-held unit allows for delivery of precise amounts of medicament to a plurality if individual animals, including fowl, in a rapid manner such that a rapid and consistent one-handed administration of the medicament or implantable device is achieved without fatigue-affected changes in the amount of medicament delivered.

### BRIEF DESCRIPTION OF THE DRAWINGS

Understanding the invention will be enhanced by referring to the accompanying drawings, in which:

FIG. 1A is a side view perspective with certain portions shown in cross section of the powered automatic injection device with the needle extended;

FIG. 1B is a side view perspective with certain portions shown in cross section of the device of FIG. 1 with the needle retracted;

FIG. 2 is a side view perspective of the hand-held unit with certain portions shown in cross section;

FIG. 3 is a side view perspective of the hand-held unit with certain portions shown in cross section; and

FIG. 4 is a side view perspective of the hand-held unit with certain portion shown in cross section.

### DETAILED DESCRIPTION OF CURRENTLY PREFERRED EMBODIMENTS

The understanding of the invention will be further enhanced by referring to the following illustrative but non-limiting examples.

The term “medicaments” is intended to include serum, vaccine, antibiotics, and other fluid products and pelletized products, such as hormones that may be used for immunizing of for treating poultry, bovine, porcine, piscine or other animals. Additionally, other pelletized products, such as those for permanent identification, may be used.

A “poxer needle” is a solid needle having an orifice formed in the side thereof.

Turning now to the drawings, in which like numbers refer to corresponding elements throughout several views. Shown is an automatic electrically powered hand-held gun-shaped injection device with a handgrip, the device of unitary construction. The injection device has an internal motor that is powered by connection to a power source, such as AC current or DC battery. Injection device acts when depressing the trigger on the injection device which initiates the internal motor to drive a helical gear that moves a fitting in response to being powered by the internal motor, the fitting limited in movement by a pair of limit switches. Injection device has a trigger on the injection device that, when depressed, activates the internal motor that powers the helical gear that in turn moves the fitting a pre-determined distance resulting in the delivery of the medicament or implantable device to the individual animal. Injection device has indicator lights that signal the operator when the injection device is ready to deliver the medicament, when medicament is being delivered, when medicament has been delivered, and when the helical gear and associated fitting is being returned to the original position readying the injection device for the next delivery of medicament. And injection device has safety switches controlled by the user that stop delivery of the medicament when that medicament is being delivered in error.

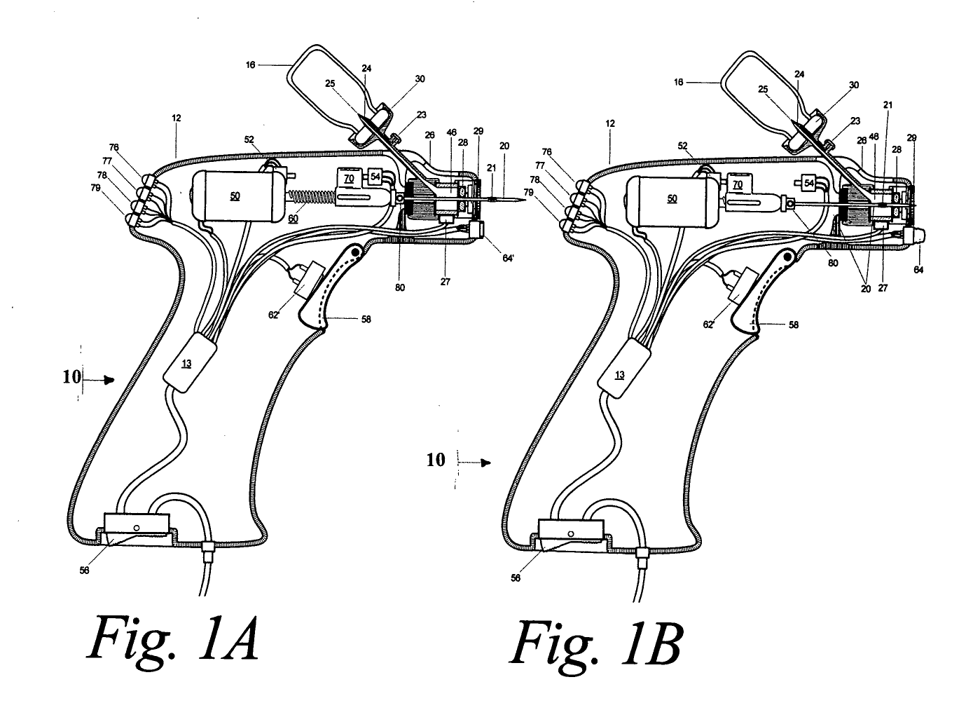

FIGS. 1A & 1B illustrates an automatic electrically powered hand-held medicament injection device 10 having a gun-shaped body 12 with a single solid needle 20. Motor 50 is an electric motor held internally to the hand-held unit that is powered by connection to a power source, such as AC current or DC battery. Because a simple motor is used, the cost of manufacturing is less than that with a computerized system. Medicament is in fluid communication between a reservoir 16 and the holding cylinder 46 through which the poxer needle 20 passes. An orifice 21, formed in the side of the poxer needle 20, receives the medicament and is pushed by the solid poxer needle 20 through the animal body part, e.g. wing of a fowl. The medicament is delivered in response to the depressing of the trigger 58. Dosage is controlled by size of the orifice 21. The poxer needle 20 is thrust such that it pierces the skin of the subject animal. When used with fowl, the poxer needle 20 would continue through the wing of the fowl leaving medicament at the wound site. The needle 20 is thrust controlled by an electric motor 50 which turns a helical gear 60 angularly which threads into a fitting 70. The fitting 70 is attached to the solid needle 20 by means of a setscrew 80. Movement of fitting 70 limits the trust of the needle 20. The movement of the fitting 70 is limited by a pair of limit switches 52, 54 that is pre-set to limit the distance the fitting 70 travels thus delivering a precise dose of medicament.

The four indicating lights display to the operator the real time status of his operation mode similarly in all examples. While the main power switch 56 is on, a holding cylinder indicating light 27 illuminates the holding cylinder 46 displaying the content of the holding cylinder 46 to the operator by the viewing port 26.

Precision dosing is controlled by the size of the orifice 21 formed in the poxer needle 20. Thrust of the poxer needle 20 is controlled by the forward limit switch 54, and the reverse limit switch 52. The distance between the two limit switches 52,54 accurately controls the poxer needle 20 thrust because they control the distance the poxer needle 20 moves. During normal operation, the needle thrust is displayed when the trigger safety switch 62 is in the depressed position, as shown in FIGS. 1A & 1B. One of the four indicating lights 76,77,78,79 illuminates displaying one of four modes to the operator delivery of the doses. Mode one is indicated by a (amber) light 76 representing the injection device is ready to deliver the medicament. A (green) light 77 indicates mode two representing the forward thrust of the delivery device; mode three is indicated by a (blue) indicating light 78 representing forward thrust is complete and ready to reverse; mode four is indicated by a (red) light 79 representing the reverse thrust of the poxer needle 20.

Mode one is commenced when the power switch 56 is on, the trigger safety switch 62 is depressed , the needle safety switch 64 is not depressed, and reverse limit switch 52 is depressed, illuminating only the (amber) indicating light 76.

Mode two is commenced when the power switch 56 is on, the trigger safety switch 62 is down, and the needle safety switch 64′ is depressed, illuminating the (green) indicating light 77, and turning off the (amber) indicating light 76.

Mode three is commenced when the power switch 56 is on, the trigger safety switch 62′ is depressed, the needle safety switch 64′ is depressed, and the forward limit switch 54 is depressed, illuminating the (blue) indicating light 78 and turning off the (green) indicating light 77.

Mode four is commenced when the power switch 56 is on, the trigger safety switch is depressed 62′, needle safety switch 64 is not depressed, and reverse stop switch 52 is not depressed, illuminating the (red) indicating light 79, and turning off the (blue) indicating light 78.

Medicament is held inside holding cylinder 46, where keeping the orifice 21 submerged in the medicament fills the orifice 21. During normal operation, medicament is supplying the holding cylinder 46 by means of a drop off fitting 30, which delivers the medicament down the fluid intake port 25. During normal operation, as the level in medicament reservoir 16 is depleted, it is replaced by air by means of an intake air port 24, which only flows inward due to an intake air valve 23.

Holding cylinder 46 is sealed by an o-ring 28, which makes a seal around the poxer needle 20. During operation, when the poxer needle 20 moves outward from the holding cylinder 46, the o-ring 28 is flexible enough not to let excess medicament out by expanding with the contours to the curves of the needle orifice while the poxer needle 20 passes by the o-ring 28. Holding cylinder 46 and the o-ring 28 are held in place by a threaded cap 29.

All wire terminations and unions are made inside a sealed junction box 13. The distance between the two limit switches 52,54 accurately controls needle thrust to render precise doses. Precision dosing and needle travel are important and is controlled during normal operation by the forward limit switch 54, and the reverse limit switch 52. These limit switches 52,54 work by reversing polarity reversing direction of movement of the fitting 70. Ultimately, the thrust of the poxer needle 20 is determined by the position of the limit switches, 52,54.

Built in safety features include: main power switch 56, which terminates any contact between an electromotive force and trigger safety switch 62; trigger safety switch 62, acts as an emergency stop button that stops all thrust of the motor 50 instantly when released; needle safety switch 64 is a switch that reverses the thrust of the poxer needle 20, when released or depressed, by changing the direction of current to the motor 50, this can occur if the device is withdrawn from the injection site at any point during injection.

FIG. 1B illustrates the injection device of FIG. 1A with the needle withdrawn into the injection device 10.

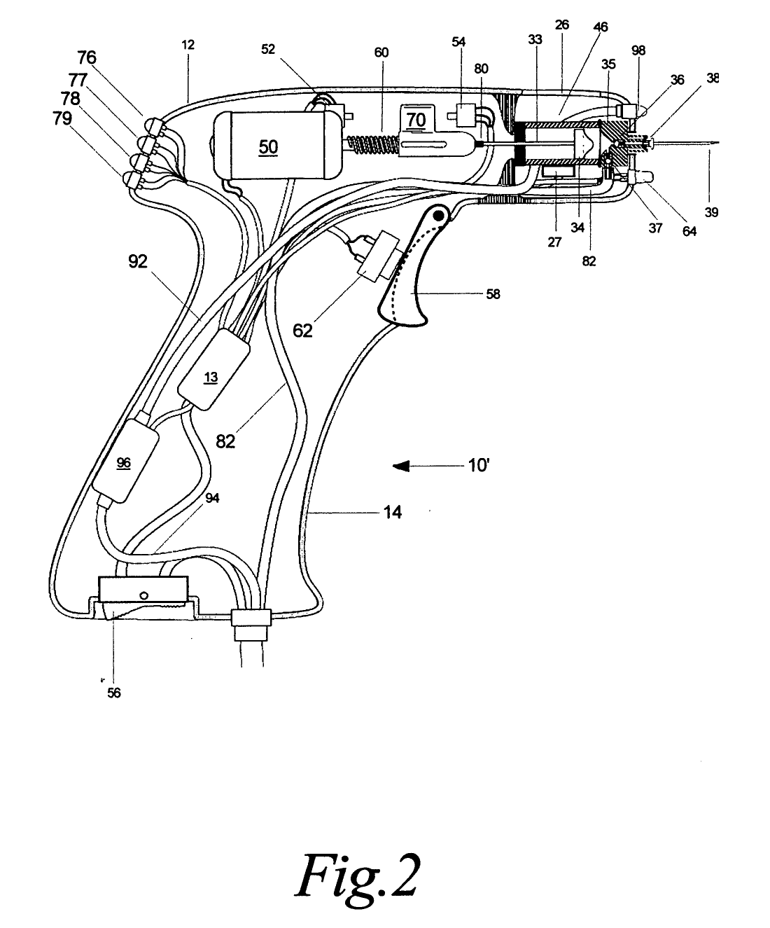

The hand-held unit 10′ is illustrated at FIG. 2 which shows an automatic electrically powered hand held medicament delivery system with a single hollow needle 39. Medicament is delivered through the hollow needle 39, by a piston 34 moving in response to the depressing of the trigger 58, causing movement of the helical gear 60 turning angularly into the fitting 70. The piston 34 is housed inside a holding cylinder 46, and is connected to fitting 70, by means of a push rod 33, and secured by a set screw 80. The piston's 34 thrust is controlled by an electric motor 50. Here the hollow needle 39 doesn't move; rather the piston held within the injection device device 10 moves pushing the medicament held in the holding chamber 46 through the hollow needle 39.

The hand-held unit 10′ again has four illuminating indicating lights to display to the operator the real time status of this operation mode. While the main power switch 56 is on, a cylinder indicating light 27, illuminates the holding cylinder 46 displaying the content of the cylinder to the operator by the viewing port 26.

Precision dosing and piston thrust is important and is stopped during normal operation by the forward limit switch 54, and the reverse limit switch 52. The distance between the two limit switches 52,54 accurately controls piston 34 thrust. During normal operation, piston 34 thrust is displayed when the trigger safety switch 62 is in the depressed position, one of the four illuminating lights illuminates displaying one of four modes to the operator assuring accurate doses. Mode one is indicated by a (amber) light 76 representing the injection device is ready to inject; a (green) light 77 indicates mode two representing the forward thrust of the piston; mode three is indicated by a (blue) indicating light 78 representing forward thrust is complete and ready to reverse; mode four is indicated by a (red) light 79 representing the reverse thrust of the piston 34. Mode one is commenced when the power switch 56 is on, the trigger safety switch 62 is depressed, the needle safety switch 64 is not depressed, and reverse stop switch 52 is depressed illuminating only the (amber) indicating light 76. Mode two is commenced when the power switch 56 is on, the trigger safety switch 62 is down, and the needle safety switch 64 is depressed, illuminating the (green) indicating light 77, and turning off the (amber) indicating light 76. Mode three is commenced when the power switch 56 is on, the trigger safety switch 62 is depressed, the needle safety switch 64 is depressed, and the forward limit switch 54 is depressed, illuminating the (blue) indicating light 78 and turning off the (green) indicating light 77. Mode four is commenced when the power switch 56 is on, the trigger safety switch 62′ is depressed, needle safety switch 64 is not depressed, and reverse stop switch 52 is not depressed, illuminating the (red) indicating light 79, and turning off the (blue) indicating light 78.

Medicament is held inside holding cylinder 46, where the flow of liquid medicament of controlled by two check valves housed in valve body 35. When the piston 34 moves in forward direction, exit valve 36 opens, and entrance valve 37 closes due to the positive pressure inside the holding cylinder 46. When the piston 34 is in the reverse direction, a negative pressure is caused inside the holding cylinder 46. Negative pressure inside the holding cylinder 46 causes entrance valve 37 to open, and exit valve 36 to close.

During normal operation, medicament is supplied to the holding cylinder 46 by means of flexible medicament supply tubing 82, which delivers the medicament from the reservoir external to the injection device to the intake valve 37 on the valve 35.

The hollow needle 39 is easily replaceable by the means of a luer lock 38 release system. The luer lock 38 secures the hollow needle 39 with the ¼ turn of the needle. The luer lock 38 functions as part of the valve 35.

All wire terminations and unions are made inside a sealed junction box 13. The distance between the two limit switches 52,54 accurately controls piston 33 thrust to render precise doses. Precision dosing and piston travel are important and is controlled during normal operation by the forward limit switch 54, and the reverse limit switch 52. Ultimately the thrust of the piston is determined by the position of the limit switches 52, 54. Built in safety features include: main power switch 56, which terminates any contact between an electromotive force and device. Trigger safety switch 62, acts as an emergency stop button that stops all thrust of the motor 50 instantly when released; needle safety switch 64 is a switch that reverses the thrust of the piston 34, when released or depressed, by changing the direction of current to the motor 50, this can occur if the device is withdrawn from the injection site at any point during injection.

The hand-held unit 10′ is equipped with an optional timed dye marking system, pump 96, by way of dye tubing 92 to dye spray port 98 to identify injections sites when the injection is completed. This is an important tool for management, for monitoring the work of injection crews.

The dye marking system is run by a pump that is turned on only while in the mode three. The dye marking system is comprised of a dye reservoir external to the injection device, entrance port on the bottom of the handle; an electric motor; a pump 96 driven by the electric motor; two tubing connections on the pump; a dye spray port 98 on the front side of the of the device 10; tubing 94 connecting exit port to pump 96 intake port; tubing 92 connecting pump exit port to dye spray port 98; a spray fitting to thread into the spray port yielding a prominent spray.

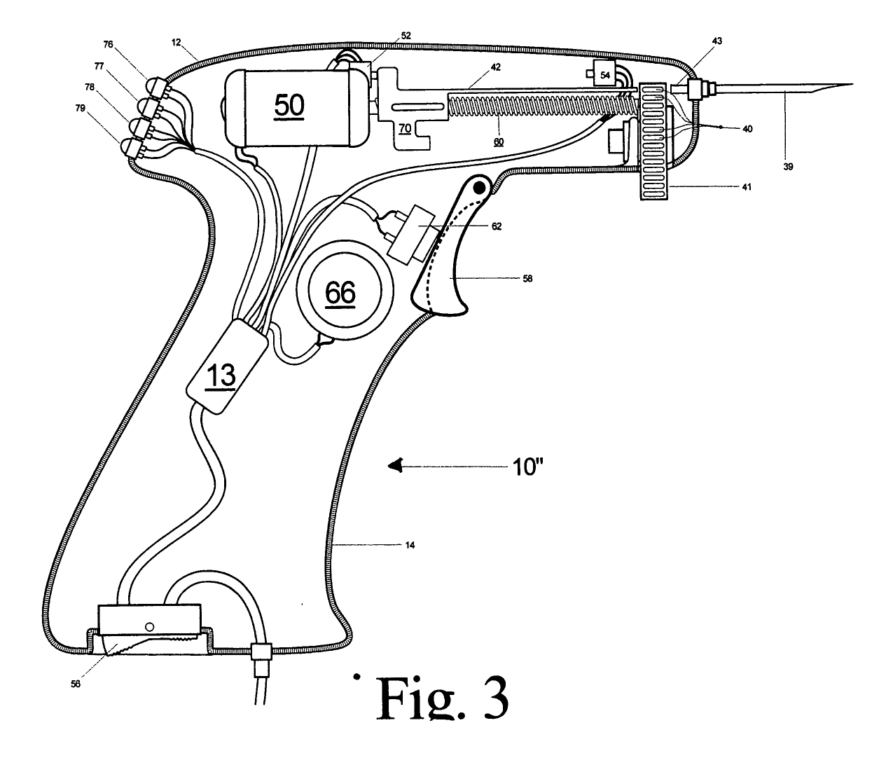

A injection device 10″ is illustrated at FIG. 3, which again is an injection device 10″ with a single hollow needle 39. Instead of delivering medicament by injection, this embodiment delivers a medicament by means of an implant 40. The implant 40 is delivered through the hollow needle 39, by a push rod 42 in response to the depressing of trigger 58. Trigger 58 is the polarity switch for motor. The push rod 42 is housed inside a guide 43, and is connected to fitting 70. The push rod 42 thrust is controlled by an electric motor 50 which turns a helical gear 60 angularly until it threads into the fitting 70. This embodiment 10″ has four indicating lights, 76,77,78,79 to display to the operator the real time status of his operation mode.

Precision dosing and push rod 42 thrust is important and is stopped during normal operation by the forward limit switch 54, and the reverse limit switch 52. The distance between the two limit switches 52, 54 accurately controls push rod 42 thrust. During normal operation, push rod 42 thrust is displayed when the trigger safety switch 62 is in the depressed position, one of the four illuminating lights illuminates displaying one of four modes to the operator assuring accurate doses. Mode one is indicated by a (amber) light 76 representing the injection device is ready to inject; a (green) light 77 indicates mode two representing the forward thrust of the pushrod; mode three is indicated by a (blue) indicating light 78 representing forward thrust is complete and ready to reverse; mode four is indicated by a (red) light 79 representing the reverse thrust of the pushrod 42. These modes operate as previously discussed.

The implant 40 is held inside a cartridge 41, where individual implants 40 are housed in separate chambers. The implant diameter is equal to or less than the diameter of the cartridge 41. The implant diameter is less than that of the hollow needle 39. The push rod 42 pushes the implant 40 out of the cartridge 41 and through the hollow needle 39 where the implant 40 exits the hollow needle 39 at the very tip. The trust of the push rod 42 does not exceed the length of the hollow needle 39. Movement of pushrod 42 is limited by limit switches 52, 54 driven by motor 50. The forward thrust of the push rod 42 causes the cartridge 41 to move to the next the holding cell prior to the push rod 42 entering the holding cell. The hollow needle 39 is easily replaceable by means of a nut to secure the hollow needle 39 to the body of the device 10″.

All wire terminations and unions are made inside a sealed junction box 13. The distance between the two limit switches 52, 54 accurately controls push rod 42 thrust to render precise doses. Precision dosing and needle travel are important and is controlled during normal operation by the forward limit switch 54, and the reverse limit switch 52.

Ultimately the thrust of the pushrod 42 is determined by the position of the control switches. Built in safety features include: main power switch 56, which terminates any contact between an electromotive force and the device; trigger safety switch 62, acts as an emergency stop button that stops all thrust of the motor instantly when released; needle safety switch 64 is a switch that reverses the thrust of the pushrod 42, when released or depressed, by changing the direction of current to the motor 50, this can occur if the device is withdrawn from the injection site at any point during injection.

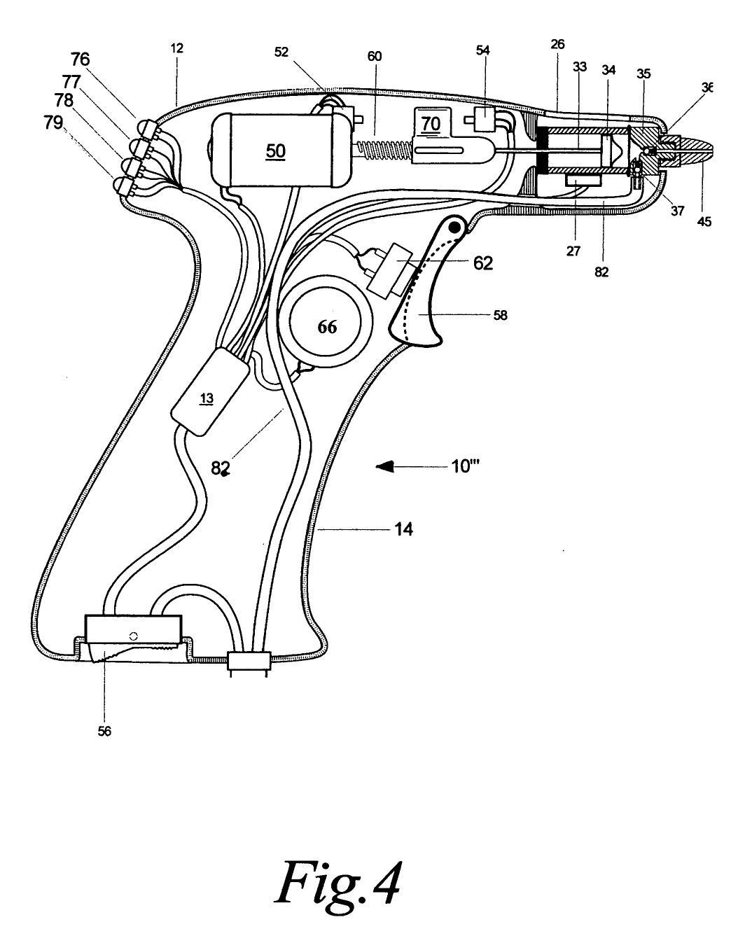

FIG. 4 illustrates a hand-held unit 10′″ of the device where an automatic electrically powered hand held medicament delivery system 10′″ includes a portable power supply and a hand held unit. In this hand-held unit 10′″, the hand held device has a single spray nozzle 45 for delivery of the medicament. Medicament is delivered through the spray nozzle 45, by force from a piston 34 in response to initiating the device by depressing the trigger 58. Trigger 58 switches the polarity of the motor again while limit switches 52, 54 limit movement of the pushrod 33. The piston 34 is housed inside a holding cylinder 46, and is connected to fitting 70, by means of a push rod 33, and secured by a set screw 80. The piston's thrust is controlled by an electric motor 50 which turns a helical gear 60 angularly which threads into the fitting 70.

This hand-held unit 10′″ again has four illuminating indicating lights, 76,77,78,79 to display to the operator the real time status of his operation mode. While the main power switch 56 is on, a holding cylinder 46 indicating light 27 illuminates the holding cylinder 46 displaying the content of the holding cylinder to the operator by the viewing port 26.

Precision dosing and piston thrust is important and is stopped during normal operation by the forward limit switch 54, and the reverse limit switch 52. The distance between the two limit switches 52,54 accurately controls piston 34 thrust. During normal operation, piston 34 thrust is displayed when the trigger safety switch 62′ is in the depressed position, one of the four illuminating lights illuminates displaying one of four modes to the operator assuring accurate doses as described above. Mode one is indicated by a (amber) light 76 representing the injection device is ready to inject; a (green) light 77 indicates mode two representing the forward thrust of the piston; mode three is indicated by a (blue) indicating light 78 representing forward thrust is complete and ready to reverse; mode four is indicated by a (red) light 79 representing the reverse thrust of the piston.

Medicament is held inside cylinder 46, where the flow of liquid medicament of controlled by two check valves housed in valve body 35. When the piston thrust is in the forward direction exit valve 36 opens, and entrance valve 37 closes due to the positive pressure inside the cylinder. When the piston 34 thrust is in the reverse direction, a negative pressure is caused inside the cylinder 46. Negative pressure inside the cylinder causes entrance valve 37 to open, and exit valve 36′ to close.

During normal operation, medicament is supplying the holding cylinder 46 by means of flexible medicament supply tubing 82 which delivers the medicament to the entrance valve 37 on the valve 35.

All wire terminations and unions are made inside a sealed junction box 13.

The distance between the two limit switches 52,54 accurately controls piston 34 thrust to render precise doses. Precision dosing and piston travel are important and is controlled during normal operation by the forward limit switch 54, and the reverse limit switch 52.

Ultimately the thrust of the piston 34 is determined by the position of the control switches 52,54. Built in safety features include: main power switch 56, which terminates any contact between an electromotive force and fourth embodiment of the device. Trigger safety switch 62, acts as an emergency stop button that stops all thrust of the motor 50 instantly when released; needle safety switch 64 is a switch that reverses the thrust of the piston 34, when released or depressed, by changing the direction of current to the motor, this can occur if the device is withdrawn from the injection site at any point during injection.

In actual use conditions, the operator holds the hand-held unit and depresses and holds trigger 58 is and the needle safety switch 64 is pressured against the body of the bird or other animal, such as the wing web, the helical gear 60 is powered by motor 50 to push the fitting 70 with the poxer needle 20 mounted thereon through holding chamber 46 such that orifice 21 formed in poxer needle 20 picks up the fluid medicament and pushes through the bird wing web depositing the medicament therein. When the operator moves the away from the body of the bird, the needle safety switch 64 automatically reverses the polarity of the motor which in turn reverses the direction of the fitting movement with the needle a follower thereto. This brings the needle back inside the hand-held unit. Limit switches 52, 54 limit the forward thrust and backward thrust of the fitting with the needle mounted thereon. When the reverse limit switch 52 is pressed by the reversing fitting 70, the polarity of the motor is switched readying it for the next injection. Because the poxer needle 20 only moves forward when both the trigger 58 and the needle safety switch 64 are both depressed, this enables the operator to continuously depress the trigger 58 and use on motion to “pole” each chicken in the wing. This device utilizes “poxer” needle, a solid needle with an orifice formed therein.

We claim:

1. A powered automatic injection device for delivery of a medicament or implant to a plurality of poultry, fish or other animals, comprising:
a) a hand-held gun-shaped injection device with a handgrip, the device of unitary construction;
b) an internal electrical motor that is powered by connection to a power source, such as AC current or DC battery;
c) means powered by said internal motor for transferring rotational motion from the electric motor into linear motion;
d) a fitting which moves in a linear plane in response to movement of said means powered by said internal motor;
e) control means for controlling the administration of one of the group including the medicament and implant, by limiting movement of said fitting;
f) medicament delivery means mounted on said fitting for delivery of one of a medicament and implant; and
g) a trigger mounted on said gun-shaped device initiates the internal motor that powers the helical gear that in turn delivers the medicament to the individual animal.

2. The injection device of claim 1, wherein said means powered by said internal motor further comprise a helical gear.

3. The injection device of claim 1, wherein said control means further comprises a pair of limit switches which limit the movement of said fitting.

4. The injection device of claim 1, wherein said medicament delivery means further comprise one of a group including a poxer needle, a hollow needle, and a spray nozzle.

5. The injection device of claim 1, further comprising indicator lights that signal the operator one of the following actions:
a) when the injection device is ready to deliver the medicament;
b) when medicament is being delivered;
c) when medicament has been delivered; and
d) when the means powered by said motor is being returned to the original position readying the injection device for the next delivery of medicament.

6. The injection device of claim 5, further comprising, safety switches, controlled by the operator, to stop delivery of the medicament when that medicament is being delivered in error.

7. The injection device of claim 1, further comprising a self-priming delivery system that automatically administers a preset dose.

8. The injection device of claim 4, wherein said fitting further comprises a push rod for pushing a pelletized medicament through a hollow needle under the skin of an animal.

9. The injection device of claim 1, further comprising means for dye marking individual animals to which medicament has been delivered.

10. A powered automatic injection device for delivery of a medicament or implant to a plurality of poultry, fish or other animals, comprising:
a) a hand-held gun-shaped injection device with a handgrip, the device of unitary construction;
b) an internal motor that is powered by connection to a power source, such as AC current or DC battery;
c) a helical gear powered by said internal motor for transferring rotational motion from the electric motor into linear motion;
d) a fitting mounted on said helical gear which moves in response to movement of said helical gear powered by the internal motor;
e) control means for controlling the administration of one of the group including the medicament and implant, by limiting movement of said fitting;
f) medicament delivery means mounted on said fitting for delivery of one of a medicament and implant; and
g) a trigger mounted on said gun-shaped device initiates the internal motor that powers the helical gear that in turn delivers the medicament to the individual animal.

11. The injection device of claim 10, wherein said control means further comprises a pair of limit switches limiting movement of said helical gear mounted fitting.

12. The injection device of claim 10, wherein said medicament delivery means further comprise one of a group including a poxer needle, a hollow needle, and a spray nozzle.

13. The injection device of claim 10, further comprising, indicator lights that signal the operator one of the following actions:
a) when the injection device is ready to deliver the medicament;
b) when medicament is being delivered;
c) when medicament has been delivered; and
d) when the helical gear is being returned to the original position readying the injection device for the next delivery of medicament.

14. The injection device of claim 10, further comprising, safety switches, controlled by the operator, to stop delivery of the medicament when that medicament is being delivered in error.

15. The injection device of claim 10, further comprising a self-priming delivery system that automatically administers a preset dose.

16. The injection device of claim 10, wherein said fitting further comprises a push rod for pushing a pelletized medicament under the skin of an animal.

17. The injection device of claim 10, further comprising means for dye marking individuals injected.

18. A powered automatic injection device for delivery of a medicament or implant to a plurality of poultry, fish or other animals, comprising:
a) a hand-held gun-shaped device with a handgrip, the device of unitary construction;
b) an internal motor that is powered by connection to a power source, such as AC current or DC battery;
c) a helical gear powered by said internal motor for transferring rotational motion from the electric motor into linear motion;
d) a fitting, mounted on said helical gear, which moves in response to movement of said helical gear powered by the internal motor;
e) a pair of limit switches which controls the administration of the medicament or implant by limiting movement of said helical gear mounted fitting;
f) medicament delivery means further comprising one of a group including a poxer needle, a hollow needle, and a spray nozzle; and
g) a trigger mounted on said gun-shaped device initiates the internal motor that powers the helical gear that in turn delivers the medicament to the individual animal.

19. The injection device of claim 18, further comprising, indicator lights that signal the operator one of the following actions:
a) when the injection device is ready to deliver the medicament;
b) when medicament is being delivered;
c) when medicament has been delivered; and
d) when the helical gear is being returned to the original position readying the injection device for the next delivery of medicament.

20. The injection device of claim 18, further comprising, safety switches, controlled by the operator, to stop delivery of the medicament when that medicament is being delivered in error.

21. The injection device of claim 18, further comprising a self-priming delivery system that automatically administers a preset dose.
